(19) United States
(12) Patent Application Publication
(10) Pub. No.: US 2024/0105347 A1
(43) Pub. Date: Mar. 28, 2024

(54) SYSTEM AND METHOD FOR ENABLING MEDICAL CONSULTATION ONLINE

(71) Applicant: SAI PRASAD GADAPA, NIZAMABAD-DIST (IN)

(72) Inventor: SAI PRASAD GADAPA, NIZAMABAD-DIST (IN)

(21) Appl. No.: 18/255,138

(22) PCT Filed: Nov. 25, 2021
(86) PCT No.: PCT/IB2021/060979

§ 371 Date: May 31, 2023

(30) Foreign Application Priority Data
Oct. 5, 2021 (IN) 202141045236
Nov. 25, 2021 (WO) PCT/IB2021/060979

(51) Int. Cl. G16H 80/00; G06Q 20/02; G06Q 30/0251; G16H 40/20; G16H 50/20

(52) U.S. Cl. CPC G16H 80/00; G06Q 20/027; G06Q 30/0261; G16H 40/20; G16H 50/20; G16H 10/60

(57) ABSTRACT

A system 10 for enabling medical consultation online is disclosed. The system includes a processing subsystem 20 which includes a registration module 50. The processing subsystem includes a specialist search module 80 to search for a first health care professional by a second health care professional. The processing subsystem includes an appointment management module 90 to reserve an appointment of the first health care professional. The processing subsystem includes a consultation module 100 to avail a consultation of first health care professional by the second health care professional. The consultation module is to provide medical records of patients relevant to the consultation. The processing subsystem includes a diagnostic module 110 to prepare a transcript of the consultation. The diagnostic module is to identify medical conditions of the patients. The diagnostic module is to recommend additional medical procedures to the patients.

### CROSS-REFERENCE TO RELATED APPLICATION

This application claims priority from a patent application filed in India having Patent Application No. 202141045236, filed on Oct. 5, 2021, and titled “SYSTEM AND METHOD FOR ENABLING MEDICAL CONSULTATION ONLINE” and a PCT Application No. PCT/IB2021/060979 filed on Nov. 25, 2021, and titled “SYSTEM AND METHOD FOR ENABLING MEDICAL CONSULTATION ONLINE.”

### FIELD OF INVENTION

Embodiments of the present disclosure relate to the field of healthcare and more particularly to a system and method for enabling medical consultation online.

### BACKGROUND

Telemedicine refers to provision of clinical services remotely through a real-time two-way communication. The real time two-way communication may be between a patient and a healthcare provider via audio and visual means. In primary care, the telemedicine may be usually in form of phone calls through which the patient may seek an advice of a doctor about non-emergency medical problems which may not require the doctor to see the patient. Telemedicine may not be considered as a replacement of face-to-face consultation, instead the telemedicine complements the face-to-face consultation. Initially, specialized videoconferencing equipments along with customized diagnostic peripherals such as stethoscopes or electrocardiogram monitors were necessary to enable telemedicine services. The healthcare provider present with the patient may use the diagnostic tools under a guidance of the doctor who may be present in a distant location, to provide vitals of the patient. With advancement in technology, now a days, computers in association with dedicated video cards are being used to provide the telemedicine services.

Even though, existing systems are offering a variety of the telemedicine services, the existing systems are mainly focusing on establishing a two-way communication channel between the patient and the doctor. Consultation of a specialist doctor may not be possible always for the patient especially when the patient is illiterate or not having sufficient knowledge about how to operate the existing systems. Further, the existing systems are not sufficient to enable the consultation of the specialist doctor by the doctor on behalf of the patient. Furthermore, the existing systems may not be able to generate a transcript of the consultation while availing the telemedicine services and are not efficient enough to support the patient by suggesting additional consultation by referring to medical records of the patient or by reminding the patient about a reserved appointment. Also, the existing systems may not be capable of recommending cancer screening, chronic disease screening and immunization to the patient.

Hence, there is a need for an improved system and method for enabling medical consultation online to address the aforementioned issue(s).

### BRIEF DESCRIPTION

In accordance with an embodiment of the present disclosure, a system for enabling medical consultation online is provided. The system includes a processing subsystem hosted on a server and configured to execute on a network to control bidirectional communications among a plurality of modules. The processing subsystem includes a registration module configured to register one or more health care professionals by creating one or more health care professional profiles in an integrated database upon receiving one or more health care professional details via one or more devices. The processing subsystem also includes a specialist search module operatively coupled to the registration module. The specialist search module is configured to conduct a search for a first health care professional in the one or more health care professional profiles created by the registration module upon receiving a search query raised by a second health care professional. The search query includes one or more parameters associated with the first healthcare professional. The processing subsystem further includes an appointment management module operatively coupled to the specialist search module. The appointment management module is configured to reserve an appointment of the first health care professional appeared in the search conducted by the specialist search module upon receiving a reservation request from the second health care professional. The processing subsystem also includes a consultation module operatively coupled to the appointment management module. The consultation module is configured to establish a two-way communication channel between the first health care professional and the second healthcare professional at a time specified during a reservation of the appointment by the appointment management module to avail a consultation of first health care professional by the second health care professional. The consultation module is also configured to provide one or more medical records of one or more patients relevant to the consultation from the integrated database to the first healthcare professional for consultation. The processing subsystem also includes a diagnostic module operatively coupled to the integrated database. The diagnostic module is configured to prepare a transcript of the consultation by analyzing one or more audio signals produced during the consultation of the first health care professional using at least one natural language processing technique. The diagnostic module is also configured to analyze the one or more medical records of the one or more patients and the transcript using a plurality of image processing techniques to generate a first pattern. The diagnostic module is further configured to compare the first pattern with a prestored data in the integrated database to identify one or more medical conditions of the one or more patients. The diagnostic module is also configured to recommend one or more additional medical procedures to the one or more patients using a plurality of neural network techniques or prestored algorithms. The one or more additional medical procedures includes at least one of an acute disease screening, chronic diseases screening, immunization programs, cancer screening or a combination thereof.

In accordance with another embodiment of the present disclosure, a method for enabling medical consultation online is provided. The method includes registering, by a registration module, one or more health care professionals by creating one or more health care professional profiles in an integrated database upon receiving one or more health care professional details via one or more devices. The method also includes conducting, by a specialist search module, a search for a first health care professional in the one or more health care professional profiles created by the registration module upon receiving a search query raised by a second health care professional. The search query comprises one or more parameters associated with the first healthcare professional. The method further includes reserving, by an appointment management module, an appointment of the first health care professional appeared in the search conducted by the specialist search module upon receiving a request of the second health care professional. The method also includes establishing, by a consultation module, a two-way communication channel between the first health care professional and the second healthcare professional at a time specified during a reservation of the appointment by the appointment management module to avail a consultation of first health care professional by the second health care professional. The method further includes providing, by the consultation module, one or more medical records of one or more patients relevant to the consultation from the integrated database to the first healthcare professional for consultation. The method also includes preparing, by a diagnostic module, a transcript of the consultation by analysing one or more audio signals produced during the consultation by the first health care professional using at least one natural language processing technique. The method also includes analysing, by the diagnostic module, the one or more medical records of the one or more patients and the transcript using a plurality of image processing techniques to generate a first pattern. The method further includes comparing, by the diagnostic module, the first pattern with a prestored data in the integrated database to identify one or more medical conditions of the one or more patients. The method also includes recommending, by the diagnostic module, one or more additional medical procedures to the one or more patients using a plurality of neural network techniques or prestored algorithms. The one or more additional medical procedures comprises at least one of an acute disease screening, chronic diseases screening, immunization programs, cancer screening or a combination thereof. To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram representation of a system for enabling medical consultation online in accordance with an embodiment of the present disclosure;

FIG. 2 is a block diagram representation of one embodiment of the system of FIG. 1 in accordance with an embodiment of the present disclosure.

FIG. 3 is a schematic representation of an exemplary embodiment of the system of FIG. 1 in accordance with an embodiment of the present disclosure;

FIG. 4 is a block diagram of a computer or a server in accordance with an embodiment of the present disclosure; and

FIG. 5(a) and FIG. 5(b) is a flow chart representing the steps involved in a method for enabling medical consultation online in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures, or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to a system and a method for enabling medical consultation online. In accordance with an embodiment of the present disclosure, a system and method for enabling medical consultation online is provided. The system includes a processing subsystem hosted on a server and configured to execute on a network to control bidirectional communications among a plurality of modules. The processing subsystem includes a registration module configured to register one or more health care professionals by creating one or more health care professional profiles in an integrated database upon receiving one or more health care professional details via one or more devices. The processing subsystem also includes a specialist search module operatively coupled to the registration module. The specialist search module is configured to conduct a search for a first health care professional in the one or more health care professional profiles created by the registration module upon receiving a search query raised by a second health care professional. The search query includes one or more parameters associated with the first healthcare professional. The processing subsystem further includes an appointment management module operatively coupled to the specialist search module. The appointment management module is configured to reserve an appointment of the first health care professional appeared in the search conducted by the specialist search module upon receiving a reservation request from the second health care professional. The processing subsystem also includes a consultation module operatively coupled to the appointment management module. The consultation module is configured to establish a two-way communication channel between the first health care professional and the second healthcare professional at a time specified during a reservation of the appointment by the appointment management module to avail a consultation of first health care professional by the second health care professional. The consultation module is also configured to provide one or more medical records of one or more patients relevant to the consultation from the integrated database to the first healthcare professional for consultation. The processing subsystem also includes a diagnostic module operatively coupled to the integrated database. The diagnostic module is configured to prepare a transcript of the consultation by analyzing one or more audio signals produced during the consultation of the first health care professional using at least one natural language processing technique. The diagnostic module is also configured to analyze the one or more medical records of the one or more patients and the transcript using a plurality of image processing techniques to generate a first pattern. The diagnostic module is further configured to compare the first pattern with a prestored data in the integrated database to identify one or more medical conditions of the one or more patients. The diagnostic module is also configured to recommend one or more additional medical procedures to the one or more patients using a plurality of neural network techniques. The one or more additional medical procedures includes at least one of an acute disease screening, chronic diseases screening, immunization programs, cancer screening or a combination thereof.

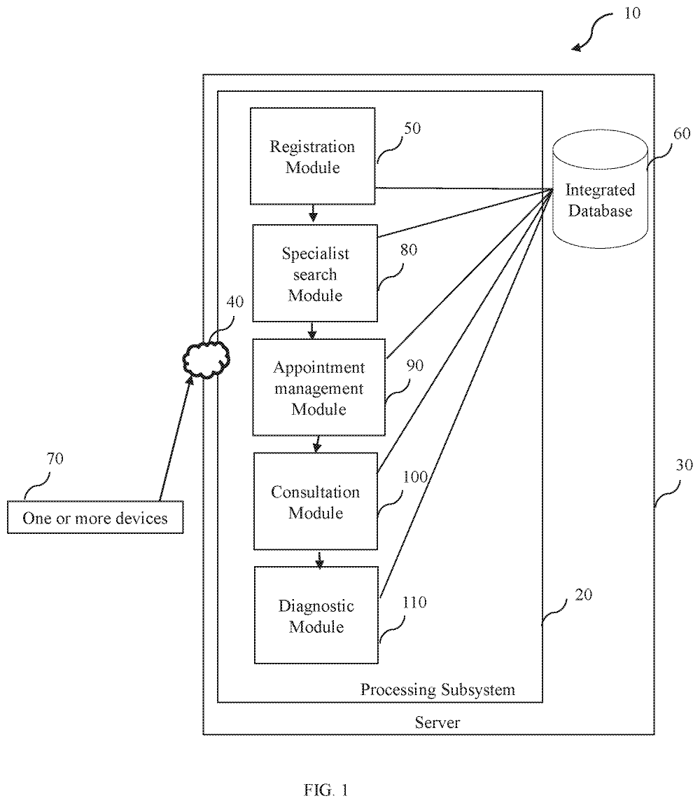

FIG. 1 is a block diagram representation of a system 10 for enabling medical consultation online in accordance with an embodiment of the present disclosure. The system 10 includes a processing subsystem 20 hosted on a server 30. In one embodiment, the server 30 may be a cloud-based server. In another embodiment, the server 30 may be a local server. The processing subsystem 20 is configured to execute on a network 40 to control bidirectional communications among a plurality of modules. In one embodiment, the network 40 may include one or more terrestrial and/or satellite networks interconnected to communicatively connect a user device to web server engine and a web crawler. In one example, the network 40 may be a private or public local area network (LAN) or wide area network (WAN), such as the Internet. In another embodiment, the network 40 may include both wired and wireless communications according to one or more standards and/or via one or more transport mediums. In one example, the network 40 may include wireless communications according to one of the 802.11 or Bluetooth specification sets, or another standard or proprietary wireless communication protocol. In yet another embodiment, the network 40 may also include communications over a terrestrial cellular network, including, a GSM (global system for mobile communications), CDMA (code division multiple access), and/or EDGE (enhanced data for global evolution) network.

The processing subsystem 20 includes a registration module 50 which is configured to register one or more health care professionals by creating one or more health care professional profiles in an integrated database 60 upon receiving one or more health care professional details via one or more devices 70. In one embodiment, the one or more health care professionals may include, but not limited to, physicians, specialists, paramedics, nurses, dental doctors and the like. In a specific embodiment, the integrated database 60 may include, but not limited to, a SQL based database, non-SQL based database, object-oriented database, hierarchical database, columnar database and the like. In some embodiments, the one or more health care professional details may include at least one of an enrollment ID, personal whereabouts, an experience, specialization and available timings for a consultation, name, email, password, phone number, one or more medical records of one or more patients being consulted and the like. In a specific embodiment, the one or more devices 70 may include a personal computer, a monitor, mobile phone, a laptop, a tablet, a personal digital assistant (PDA) or the like. In one embodiment, the integrated database 60 may store documents, prestored data, historic data and the like.

The processing subsystem 20 also includes a specialist search module 80 operatively coupled to the registration module 50. The specialist search module 80 is configured to conduct a search for a first health care professional in the one or more health care professional profiles created by the registration module 50 upon receiving a search query raised by a second health care professional. The search query includes one or more parameters associated with the first healthcare professional. In one embodiment, the one or more parameters may include at least one of a specialization, the available timings for the consultation, the experience, name, email, location or a combination thereof. In an exemplary embodiment, the search for the first health care professional in the one or more health care professional profiles corresponding to the search query raised by the second health care professional may be based on a representational state transfer technique. The processing subsystem 20 further includes an appointment management module 90 operatively coupled to the specialist search module 80. The appointment management module 90 is configured to reserve an appointment of the first health care professional appeared in the search conducted by the specialist search module 80 upon receiving a reservation request from the second health care professional. In a specific embodiment, the appointment management module 90 may enable the first health care professional to update details such as working hours, available timings on an online platform. In one embodiment, the appointment management module 90 may enable the second health care professional to reserve, modify, and cancel the appointment of the first health care professional based on the details updated by the first health care professional on the online platform. In some embodiments, the online platform may notify the first health care professional regarding the appointments reserved, modification of the appointments, or the cancellation of the appointments.

The processing subsystem 20 also includes a consultation module 100 operatively coupled to the appointment management module 90. The consultation module 100 is configured to establish a two-way communication channel between the first health care professional and the second healthcare professional at a time specified during a reservation of the appointment by the appointment management module 90 to avail a consultation of first health care professional by the second health care professional. In one embodiment, the two-way communication channel may support visual communication, verbal communication, written communication or a combination thereof. In a specific embodiment, the consultation of first healthcare professional by the second health care professional may be on behalf of one or more patients being under treatment by the second health care professional. The consultation module 100 is also configured to provide one or more medical records of one or more patients relevant to the consultation from the integrated database 60 to the first healthcare professional for consultation. In one embodiment, the one or more medical records of the one or more patients may include, but not limited to admission notes, on-service notes, progress notes (SOAP notes), preoperative notes, operative notes, postoperative notes, procedure notes, delivery notes, postpartum notes, and discharge notes and the like. In some embodiments, the one or more medical records may be in different formats such as jpg, doc, pdf and the like.

The processing subsystem 20 also includes a diagnostic module 110 operatively coupled to the integrated database 60. The diagnostic module 110 is configured to prepare a transcript of the consultation by analyzing one or more audio signals produced during the consultation of the first health care professional using at least one natural language processing technique, wherein the one or more audio signals are corresponding to speech of conversation of the first health care professional with the second health care professional. In one embodiment, the natural language processing technique may include, named entity recognition, sentiment analysis, text summarization, aspect mining, topic modeling and the like. Initially, the one or more audio signals produced during the consultation may be converted into text. A semantic analysis may be carried out to understand a meaning of the text. After the semantic analysis, a sentiment analysis may be performed to detect a positive or negative sentiment in the text. Output of the semantic analysis and the sentiment analysis may be collectively used by the diagnostic module 110 to prepare the transcript of the consultation.

The diagnostic module 110 is also configured to analyze the one or more medical records of the one or more patients and the transcript using a plurality of image processing techniques to generate a first pattern. In one embodiment, the image processing techniques may be used to generate bounding boxes and segmentation masks for one or more regions detected on the one or more medical records and the transcript using a feature pyramid network technique. In one embodiment, the bounding boxes may include a surrounding sphere (SS), an axis-aligned bounding box (AABB), an oriented bounding box (OBB), a fixed-direction hull (FDH), and a convex hull (CH) and the like. In some embodiments, the segmentation masks may be based on semantic segmentation, instance segmentation and the like. As used herein, the one or more regions may be defined as a group of connected pixels with similar properties. As used herein, the feature pyramid network technique may be defined as a feature extractor which takes a single-scale image of an arbitrary size as input, and outputs proportionally sized feature maps at multiple levels, in a fully convolutional fashion. In one embodiment, the first pattern corresponding to the the one or more medical records of the one or more patients and the transcript may be generated based on the bounding boxes and segmentation masks. In one embodiment, the feature pyramid network technique may be replaced by a mask region based convolutional neural network or a graph region based convolutional neural network technique.

The diagnostic module 110 is further configured to compare the first pattern with a prestored data in the integrated database 60 to identify one or more medical conditions of the one or more patients. In one embodiment, the prestored data may include, but not limited to, X-ray images, medical reports, diagnostic reports, images of healthy and diseased body parts and the like. In some embodiments, the one or more medical conditions of the one or more patients may include, but not limited to, diabetes, heart diseases, cancer, lung diseases and the like. The diagnostic module 110 is also configured to recommend one or more additional medical procedures to the one or more patients using a plurality of neural network techniques. The one or more additional medical procedures includes at least one of an acute disease screening, chronic diseases screening, immunization programs, cancer screening or a combination thereof.

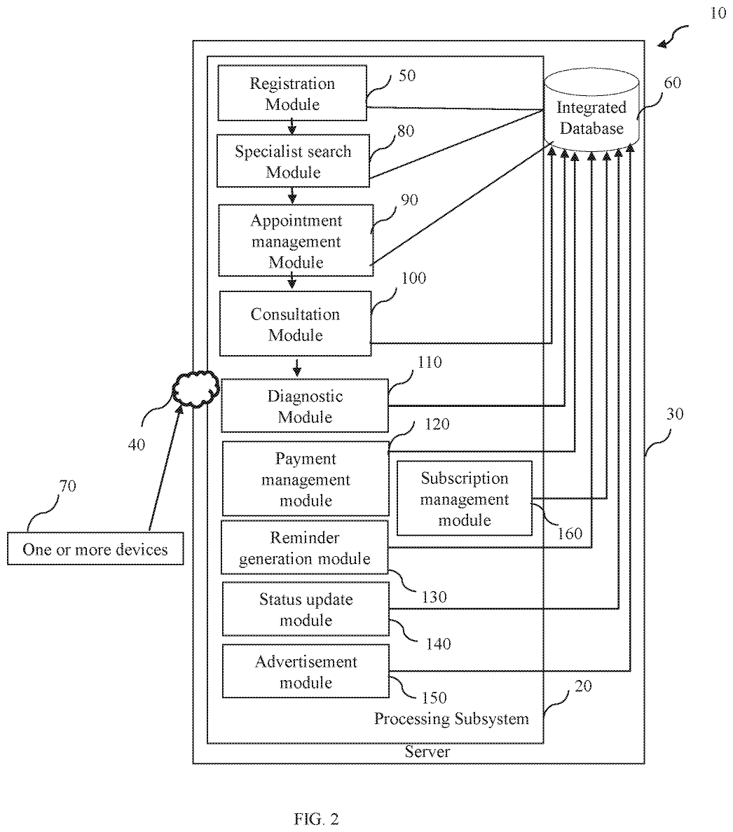

FIG. 2 is a block diagram representation of one embodiment of the system 10 of FIG. 1 in accordance with an embodiment of the present disclosure. The system 10 of FIG. 1 includes the registration module 50, the specialist search module 80, the appointment management module 90, the consultation module 100, the diagnostic module 110. In one embodiment, the system 10 of FIG. 1 may include the processing subsystem 20 including a payment management module 120. The payment management module 120 may be configured to generate a payment link to the second health care professional to enable a payment of the predefined consultation fee. The payment management module 120 may also be configured to enable the payment of the predefined consultation fee via the payment link through one or more payment gateways. In one embodiment, the payment management module may avail the predefined consultation fee to the first health care professional only after the consultation with the second health care professional. In one embodiment, the payment of the predefined consultation fee may be done in various modes such as via debit card, credit card, online banking, wallet payment, internet banking and the like. The payment management module 120 may further be configured to generate a payment receipt upon successful payment of the predefined consultation fee and update the payment details corresponding to the one or more healthcare professional profiles. In one embodiment, the payment management module may initiate a refund to the second health care professional upon cancelling the reservation of the appointment. Consider a scenario in which the first health care professional is seeking an appointment of the second health care professional. In order to reserve the appointment of the second health care professional, the first health care professional may navigate to a profile of the second health care professional and may check for an available slot of the second healthcare professional. The appointment of the second health care professional may be reserved by the first healthcare professional by choosing the available slot and by paying the predefined consultation fee by clicking on a payment button embedded in the profile of the second health care professional. The first health care professional may pay the predefined consultation fee by various means such as via online banking, upi payment, debit card, credit card and the like. The first healthcare professional may get a receipt of the payment receipt upon successful payment of the predefined consultation fee and the payment details may be updated in corresponding the one or more healthcare professional profiles.

In one embodiment, the processing subsystem 20 may also include a reminder generation module 130 operatively coupled to the integrated database 60. The reminder generation module 130 may be configured to generate one or more alerts to remind the one or more health care professionals regarding the appointment based on the time specified during the reservation of the appointment by the appointment management module 90. Consider a scenario in which the first health care professional reserved the appointment of the second health care professional by choosing the available slot of the second healthcare professional along with payment of the predefined consultation fee. In such a scenario the reminder generation module 130 may generate one or more alerts to the first health care professional and the second health care professional regarding the appointment based on the time specified during the reservation of the appointment by the appointment management module 90.

In one embodiment, the processing subsystem 20 may include a status update module 140 operatively coupled to the integrated database 60. The status update module 140 may be configured to update one or more available timings for consultation by the one or more health care professionals in corresponding the one or more health care professional profiles. The status update module 140 may also be configured to update a real time availability of the one or more healthcare professionals corresponding to the one or more health care professional profiles. Consider a scenario in which the second health care professional may be looking forward to updating available slots as a reference to the first healthcare professional to reserve the appointment of the second healthcare professional. The status update module 140 may enable the second health care professional to update the available slots in the second health care professional profile upon receiving on or more inputs from the second health care professional such as time, duration of availability, and the like. The second healthcare professional may switch between different statuses such as online, offline, idle, to intimate the first healthcare professional regarding an availability of the second healthcare professional in real-time.

In a specific embodiment, the processing subsystem 20 may include an advertisement module 150 operatively coupled to the integrated database 60. The advertisement module 150 may be configured to display one or more advertisements on the one or more devices 70 associated with one or more healthcare professionals upon receiving one or more targeting parameters from the integrated database 60 and geo location data associated with the one or more healthcare professionals. In one embodiment, the one or more targeting parameters may include, but not limited to, age, sex, region, personal preferences and the like. In one embodiment, one or more URLs may be embedded in the one or more advertisements to redirect the one or more health care professionals to a predefined platform upon interacting with the one or more advertisements. Consider a scenario in which the advertisement module 150 is configured to display one or more advertisements regarding a medicine which launched recently. The manufacture of the medicine is planning to reach the one or more health care professionals treating a specific disease in a specific region. The advertisement module 150 may be able to short list a group of health care professionals from the one or more heath care professionals based on predefined criteria to display one or more advertisements regarding the medicine. The predefined criteria may include, but not limited to the one or more health care professionals treating the specific disease, the specific region, a specific age group and the like.

In some embodiments, the processing subsystem 20 may include a subscription management module 160 operatively coupled to the integrated database 60. The subscription management module 160 may be configured to provide controlled access for the one or more healthcare professionals to the plurality of the modules based on a subscription secured by the one or more healthcare professionals. Consider a scenario in which the first health care professional may have a basic subscription and the second health care professional may have a premium subscription. The first health care professional may be able to perform various operations such as searching for the second health care professional, reserving the appointment of the second health care professional upon payment of the predefined consultation fee, avail the consultation of the second health care professional and the like. In detail, the premium subscription secured by the second health care professional enables the second healthcare professional to receive the appointment request from the first health care professional. On the other hand, the first health care professional may not be able to receive the appointment request from a second healthcare professional since the subscription secured by the first health care professional is the basic subscription.

Consider another example in which the subscription management module 160 may enable a first organization to enroll the one or more health care professionals by providing one or more details such as name, specialty, registration number and the like. A subscription fee for enrolling the one or more health care professionals may be paid by the first organization. After registration, the one or more health care professionals may access the plurality of the modules in two ways such as an individual or as an employee of the first organization. Accessing the plurality of the modules as the employee of the first organization enables the first health care professional to connect with the one or more health care professionals working in the first organization. Accessing the plurality of the modules as the individual enables the first health care professional to connect with the one or more health care professionals working in other organizations.

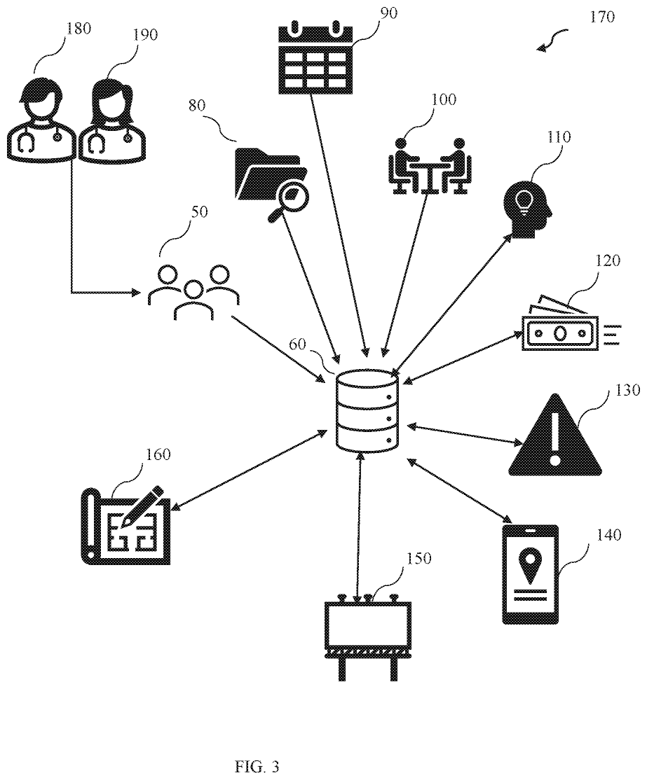

FIG. 3 is a schematic representation of an exemplary embodiment 170 of the system 10 of FIG. 1 in accordance with an embodiment of the present disclosure. Consider an example of two health care professionals such as professional A 180 and professional B 190. The registration module 50 may create individual profiles for the professional A 180 and the professional B 190 in the integrated database 60 upon receiving some details from the professional A 180 and the professional B 190 via mobile phones possessed by the professional A 180 and the professional B 190. The details may include, but not limited to an enrollment number, name, address, phone number, experience, specification, consultation histories and the like. Suppose the professional A 180 is a physician and the professional B 190 is a pulmonologist. The pulmonologist is a specialist who specializes in respiratory system. Consider a situation in which a patient C (not shown in FIG. 3) is seeking medical assistance of the professional A 180 regarding some breathing difficulties. The professional A 180 may examine the patient C (not shown in FIG. 3) and suggest some additional diagnosis such as X-ray and scanning. By examining results of the X-ray and scanning, the professional A 180 decides to seek advice from the professional B 190 regarding a medical condition of the patient C (not shown in FIG. 3). The professional A 180 may search for a medical practitioner based on certain parameters such as specialization in respiratory system. The specialist search module 80 may conduct the search for the medical practitioner in the integrated database 60 according to the certain parameters mentioned by the professional A 180. The individual profile of the professional B 190 may appear in the search conducted by the specialist search module 80. The professional A 180 may look for one or more details in the individual profile of the professional B 190. The one or more details may include, but not limited to, an enrollment ID of the professional B 190, available timings for consultation, consultation fee, experience and the like. The professional A 180 may reserve an appointment of the professional B 190 by choosing an available slot displayed in the profile of the professional B 190. The professional A 180 may also have to pay the consultation fee for reserving the appointment of the professional B 190. The appointment management module 90 may reserve the appointment of the professional B 190 upon paying the consultation fee by the professional A 180 using a suitable payment method. The payment management module 120 may enable payment of the consultation fee by generating a payment link and a payment receipt may be issued to the professional A 180 upon successfully completing the payment of the consultation fee. The reminder generation module 130 may alert the professional A 180 and the professional B 190 regarding the appointment prior to the appointment. The consultation module 100 may establish a two-way communication channel between the professional A 180 and the professional B 190 at the time specified during the reservation of the appointment. The two-way communication may be audio based, text based, video based or a combination thereof. The professional A 180 may provide relevant medical records of the patient to the professional B 190 during the consultation. The relevant medical records may include the results of the X-ray and the scanning. The professional B 190 may give advice to the professional A 180 regarding treatment procedures for treating the patient C (not shown in FIG. 3). The diagnostic module 110 may prepare the transcript of the consultation using at least one natural language processing techniques. Further, the diagnostic module 110 may analyze the medical records and the transcript using image processing techniques to generate the first pattern related to the medical conditions of the patient C (not shown in FIG. 3). The diagnostic module 110 may analyze the results of the X-Ray and scanning using image processing techniques to identify one or more aspects of a human body and related textures to generate the first pattern. The one or more aspects of the human body may include, but not limited to, organs, tissues, muscles, blood vessels and the like. The diagnostic module 110 may compare the first pattern with the prestored data in the integrated database 60 to identify the one or more medical conditions of the patient C (not shown in FIG. 3). The prestored data may include but not limited to images of healthy organs, medical reports, diagnostic reports and the like. Suppose the patient C (not shown in FIG. 3) is having swollen lungs. Comparison of the results of the X-ray and scanning of the patient C (not shown in FIG. 3) with the results of the X-ray and scanning of a healthy counterpart may reveal about the swollen lungs of the Patient C (not shown in FIG. 3). The diagnostic module 110 may further suggest treatments for the patient C (not shown in FIG. 3) according to the one or more medical conditions identified. The status update module 140 may update the availability of the professional B 190 in the profile of the professional B 190. The status update module 140 may also update a real time availability of the professional B 190. The advertisement module 150 may display one or more advertisements in the one or more device based on the one or more targeting parameters and geolocation obtained from the integrated database 60. The one or more advertisements may include, but not limited to advertisements related medicines, medical equipment, commercial products and the like. The one or more targeting parameters may include, but not limited to, specialty of the professional A 180 or professional B 190, experience, region and the like. The subscription management module 160 may provide controlled access for the professional A 180 and the professional B 190 to various modules. Suppose the professional A 180 has a premium subscription and the professional B 190 has a basic subscription. The professional A 180 may access any modules at any time without any restrictions. Since professional B 190 is having a basic subscription access to specific modules is restricted for the professional B 190.

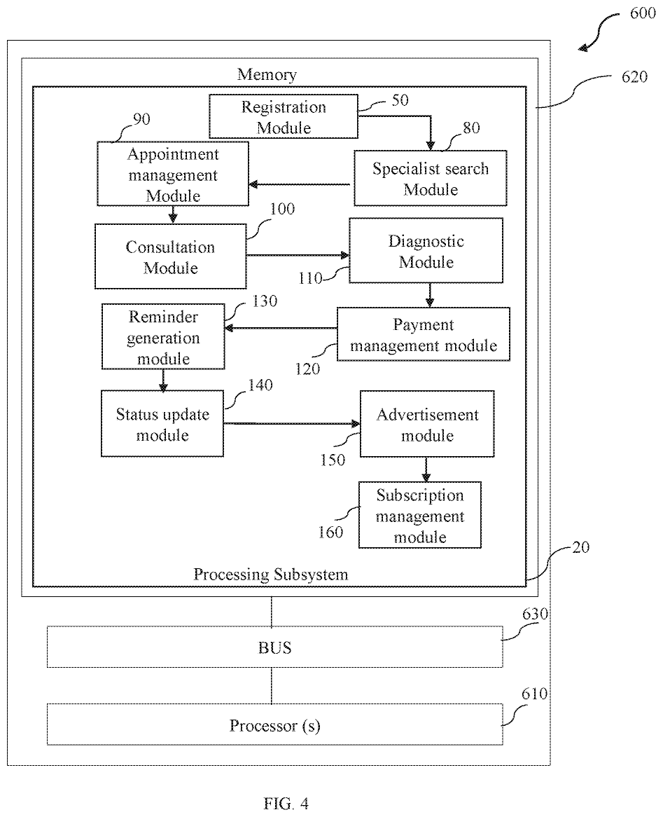

FIG. 4 is a block diagram of a computer 600 or a server in accordance with an embodiment of the present disclosure. The server 600 includes processors 610, and memory 620 operatively coupled to the bus 630. The processors 610, as used herein, includes any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The memory 620 includes several subsystems stored in the form of executable program which instructs the processor to perform the method steps illustrated in FIG. 1. The memory 620 is substantially similar to system 10 of FIG. 1. The memory 620 has the following subsystems: a processing subsystem 20 including a registration module 50, specialist search module 80, appointment management module 90, consultation module 100, diagnostic module 110, payment management module 120, reminder generation module 130, status update module 140, advertisement module 150, subscription management module 160. The plurality of modules of the processing subsystem 20 performs the functions as stated in FIG. 1 and FIG. 2. The bus 630 as used herein refers to be the internal memory channels or computer network that is used to connect computer components and transfer data between them. The bus 630 includes a serial bus or a parallel bus. The serial bus transmit data in bit-serial format and the parallel bus transmit data across multiple wires. The bus 630 as used herein, may include but not limited to, a system bus, an internal bus, an external bus, an expansion bus, a frontside bus, a backside bus, and the like.

The processing subsystem 20 includes a registration module 50 configured to register one or more health care professionals by creating one or more health care professional profiles in an integrated database (FIG. 1, 60) upon receiving one or more health care professional details via one or more devices 70. The processing subsystem 20 also includes a specialist search module 80 operatively coupled to the registration module 50. The specialist search module 80 is configured to conduct a search for a first health care professional in the one or more health care professional profiles created by the registration module 50 upon receiving a search query raised by a second health care professional. The search query includes one or more parameters associated with the first healthcare professional. The processing subsystem 20 further includes an appointment management module 90 operatively coupled to the specialist search module 80. The appointment management module 90 is configured to reserve an appointment of the first health care professional appeared in the search conducted by the specialist search module 80 upon receiving a reservation request from the second health care professional. The processing subsystem 20 also includes a consultation module 100 operatively coupled to the appointment management module 90. The consultation module 100 is configured to establish a two-way communication channel between the first health care professional and the second healthcare professional at a time specified during a reservation of the appointment by the appointment management module 90 to avail a consultation of first health care professional by the second health care professional. The consultation module 100 is also configured to provide one or more medical records of one or more patients relevant to the consultation from the integrated database 60 to the first healthcare professional for consultation. The processing subsystem 20 also includes a diagnostic module 110 operatively coupled to the integrated database 60. The diagnostic module 110 is configured to prepare a transcript of the consultation by analyzing one or more audio signals produced during the consultation of the first health care professional using at least one natural language processing technique. The diagnostic module 110 is also configured to analyze the one or more medical records of the one or more patients and the transcript using a plurality of image processing techniques to generate a first pattern. The diagnostic module 110 is further configured to compare the first pattern with a prestored data in the integrated database (FIG. 1, 60) to identify one or more medical conditions of the one or more patients. The diagnostic module 110 is also configured to recommend one or more additional medical procedures to the one or more patients using a plurality of neural network techniques. The one or more additional medical procedures includes at least one of an acute disease screening, chronic diseases screening, immunization programs, cancer screening or a combination thereof.

The processing subsystem 20 also includes a payment management module 120. The payment management module 120 is configured to generate a payment link to the second health care professional to enable a payment of the predefined consultation fee. The payment management module 120 is also configured to enable the payment of the predefined consultation free via the payment link through one or more payment gateways. The payment management module 120 is further configured to generate a payment receipt upon successful payment of the predefined consultation fee and update the payment details corresponding to the one or more healthcare professional profiles. The processing subsystem 20 also includes a reminder generation module 130. The reminder generation module 130 is configured to generate one or more alerts to remind the one or more health care professionals regarding the appointment based on the time specified during the reservation of the appointment by the appointment management module 90. The processing subsystem 20 further includes a status update module 140. The status update module 140 is configured to update one or more available timings for consultation by the one or more health care professionals in corresponding the one or more health care professional profiles, The status update module 140 is also configured to update a real time availability of the one or more healthcare professionals corresponding to the one or more health care professional profiles. The processing subsystem 20 also includes an advertisement module 150 operatively coupled to the integrated database 60. The advertisement module 150 is configured to display one or more advertisements on the one or more devices 70 associated with one or more healthcare professionals upon receiving one or more targeting parameters from the integrated database 60 and geo location data associated with the one or more healthcare professionals. The processing subsystem 20 further includes a subscription management module 160 operatively coupled to the integrated database 60. The subscription management module 160 is configured to provide controlled access for the one or more healthcare professionals to the plurality of the modules based on a subscription secured by the one or more healthcare professionals

Computer memory elements may include any suitable memory device(s) for storing data and executable program, such as read only memory, random access memory, erasable programmable read only memory, electrically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the processor(s) 610.

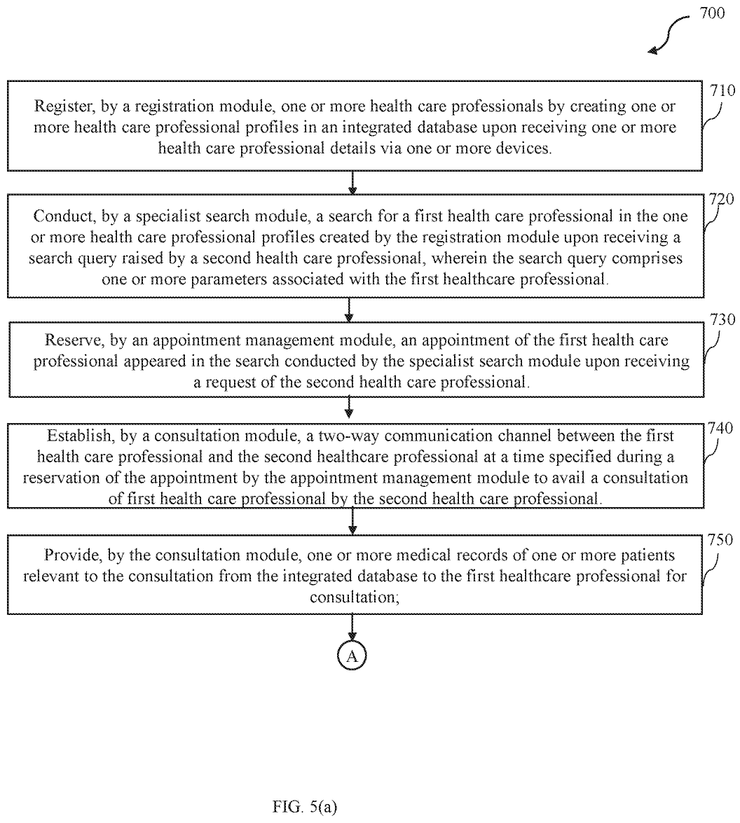

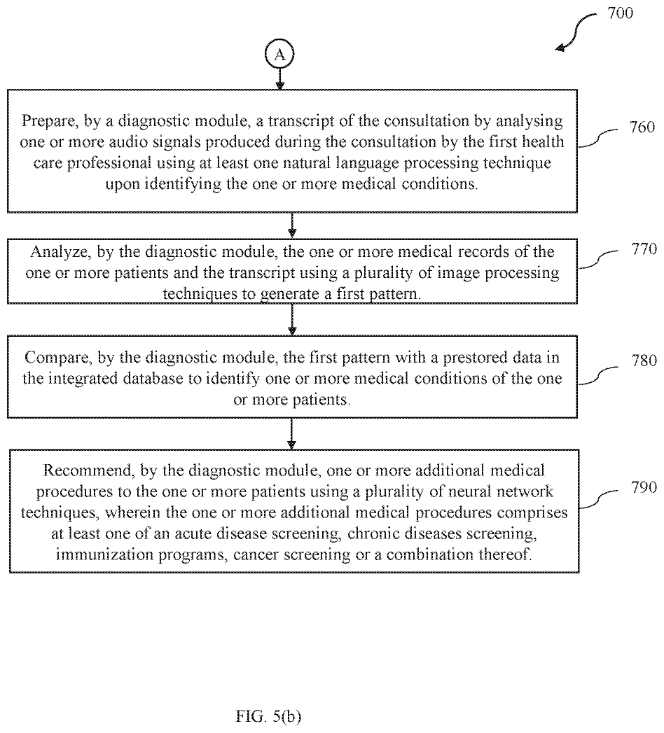

FIG. 5(a) and FIG. 5(b) is a flow chart representing the steps involved in a method 700 for enabling medical consultation online in accordance with an embodiment of the present disclosure. The method 700 includes registering one or more health care professionals by creating one or more health care professional profiles in an integrated database upon receiving one or more health care professional details via one or more devices in step 710. In one embodiment, registering one or more health care professionals by creating one or more health care professional profiles in an integrated database upon receiving one or more health care professional details via one or more devices includes registering one or more health care professionals by creating one or more health care professional profiles in an integrated database upon receiving one or more health care professional details via one or more devices by a registration module. In one embodiment, the one or more health care professionals may include, but not limited to, physicians, specialists, paramedics, nurses, dental doctors and the like. In a specific embodiment, the integrated database may include, but not limited to, a SQL based database, non-SQL based database, object-oriented database, hierarchical database, columnar database and the like. In some embodiments, the one or more health care professional details may include at least one of an enrollment ID, personal whereabouts, an experience, specialization and available timings for a consultation, one or more medical records of one or more patients being consulted and the like. In a specific embodiment, the one or more devices may include a personal computer, a monitor, mobile phone, a laptop, a tablet, a personal digital assistant (PDA) or the like. In one embodiment, the integrated database may store documents, prestored data, historic data and the like.

The method 700 also includes conducting a search for a first health care professional in the one or more health care professional profiles created by the registration module upon receiving a search query raised by a second health care professional. The search query comprises one or more parameters associated with the first healthcare professional in step 720. In one embodiment, conducting a search for a first health care professional in the one or more health care professional profiles created by the registration module upon receiving a search query raised by a second health care professional, wherein the search query comprises one or more parameters associated with the first healthcare professional includes conducting a search for a first health care professional in the one or more health care professional profiles created by the registration module upon receiving a search query raised by a second health care professional, wherein the search query comprises one or more parameters associated with the first healthcare professional by a specialist search module. In one embodiment, the one or more parameters may include at least one of a specialization, the available timings for the consultation, the experience or a combination thereof.

The method 700 also includes reserving an appointment of the first health care professional appeared in the search conducted by the specialist search module upon receiving a request of the second health care professional in step 730. In one embodiment, reserving an appointment of the first health care professional appeared in the search conducted by the specialist search module upon receiving a request of the second health care professional includes reserving an appointment of the first health care professional appeared in the search conducted by the specialist search module upon receiving a request of the second health care professional by an appointment module.

The method 700 further includes establishing a two-way communication channel between the first health care professional and the second healthcare professional at a time specified during a reservation of the appointment by the appointment management module to avail a consultation of first health care professional by the second health care professional in step 740. In one embodiment, establishing a two-way communication channel between the first health care professional and the second healthcare professional at a time specified during a reservation of the appointment by the appointment management module to avail a consultation of first health care professional by the second health care professional includes establishing a two-way communication channel between the first health care professional and the second healthcare professional at a time specified during a reservation of the appointment by the appointment management module to avail a consultation of first health care professional by the second health care professional by a consultation module. In one embodiment, the two-way communication channel may support visual communication, verbal communication, written communication or a combination thereof. In a specific embodiment, the consultation of first healthcare professional by the second health care professional may be on behalf of one or more patients being under treatment by the second health care professional.

The method 700 also includes providing one or more medical records of one or more patients relevant to the consultation from the integrated database to the first healthcare professional for consultation in step 750. In one embodiment, providing one or more medical records of one or more patients relevant to the consultation from the integrated database to the first healthcare professional for consultation includes providing one or more medical records of one or more patients relevant to the consultation from the integrated database to the first healthcare professional for consultation by the consultation module. In one embodiment, the one or more medical records of the one or more patients may include, but not limited to admission notes, on-service notes, progress notes (SOAP notes), preoperative notes, operative notes, postoperative notes, procedure notes, delivery notes, postpartum notes, and discharge notes and the like.

The method 700 further includes preparing, by a diagnostic module, a transcript of the consultation by analyzing one or more audio signals produced during the consultation by the first health care professional using at least one natural language processing technique upon identifying the one or more medical conditions in step 760. In one embodiment, preparing, by a diagnostic module, a transcript of the consultation by analyzing one or more audio signals produced during the consultation by the first health care professional using at least one natural language processing technique upon identifying the one or more medical conditions includes preparing, by a diagnostic module, a transcript of the consultation by analyzing one or more audio signals produced during the consultation by the first health care professional using at least one natural language processing technique upon identifying the one or more medical conditions by a diagnostic module. In one embodiment, the natural language processing technique may include, named entity recognition, sentiment analysis, text summarization, aspect mining, topic modeling and the like. Initially, the one or more audio signals produced during the consultation may be converted into text. A semantic analysis may be carried out to understand a meaning of the text. After the semantic analysis, a sentiment analysis may be performed to detect a positive or negative sentiment in the text. Output of the semantic analysis and the sentiment analysis may be collectively used by the diagnostic module to prepare the transcript of the consultation.

The method 700 also includes analyzing the one or more medical records of the one or more patients and the transcript using a plurality of image processing techniques to generate a first pattern in step 770. In one embodiment, analyzing the one or more medical records of the one or more patients and the transcript using a plurality of image processing techniques to generate a first pattern includes analyzing the one or more medical records of the one or more patients and the transcript using a plurality of image processing techniques to generate a first pattern by the diagnostic module. In one embodiment, the image processing techniques may be used to generate bounding boxes and segmentation masks for one or more regions detected on the one or more medical records and the transcript using a feature pyramid network technique. In one embodiment, the bounding boxes may include a surrounding sphere (SS), an axis-aligned bounding box (AABB), an oriented bounding box (OBB), a fixed-direction hull (FDH), and a convex hull (CH) and the like. In some embodiments, the segmentation masks may be based on semantic segmentation, instance segmentation and the like. As used herein, the one or more regions may be defined as a group of connected pixels with similar properties. As used herein, the feature pyramid network technique may be defined as a feature extractor which takes a single-scale image of an arbitrary size as input, and outputs proportionally sized feature maps at multiple levels, in a fully convolutional fashion. In one embodiment, the first pattern corresponding to the the one or more medical records of the one or more patients and the transcript may be generated based on the bounding boxes and segmentation masks. In one embodiment, the feature pyramid network technique may be replaced by a mask region based convolutional neural network or a graph region based convolutional neural network technique.

The method 700 further includes comparing the first pattern with a prestored data in the integrated database to identify one or more medical conditions of the one or more patients in step 780. In one embodiment, comparing the first pattern with a prestored data in the integrated database to identify one or more medical conditions of the one or more patients includes comparing the first pattern with a prestored data in the integrated database to identify one or more medical conditions of the one or more patients by the diagnostic module. In one embodiment, the prestored data may include, but not limited to, X-ray images, medical reports, diagnostic reports, images of healthy and diseased body parts and the like. In some embodiments, the one or more medical conditions of the one or more patients may include, but not limited to, diabetes, heart diseases, cancer, lung diseases and the like.

The method 700 also includes recommending one or more additional medical procedures to the one or more patients using a plurality of neural network techniques, wherein the one or more additional medical procedures comprises at least one of an acute disease screening, chronic diseases screening, immunization programs or a combination thereof in step 790. In one embodiment, recommending one or more additional medical procedures to the one or more patients using a plurality of neural network techniques, wherein the one or more additional medical procedures comprises at least one of an acute disease screening, chronic diseases screening, immunization programs or a combination thereof includes recommending one or more additional medical procedures to the one or more patients using a plurality of neural network techniques, wherein the one or more additional medical procedures comprises at least one of an acute disease screening, chronic diseases screening, immunization programs, cancer screening or a combination thereof by the diagnostic module.

Various embodiments of the system and method for enabling medical consultation online described above enable various advantages. The consultation module realizes the two-way communication channel between the first health care professional and the second health care professional on behalf of the patient, enables addressal of concerns of the patient in an efficient manner especially when the patient is illiterate or not having sufficient knowledge about how to utilize the tele medicine. Further, the diagnostic module prepares the transcript of the consultation and also recommends additional consultations to the patient by referring the medical records of the patient. Furthermore, the reminder generation module keeps track of the appointments and intimates the one or more healthcare professionals prior to the appointments.

It will be understood by those skilled in the art that the foregoing general description and the following detailed description are exemplary and explanatory of the disclosure and are not intended to be restrictive thereof. While specific language has been used to describe the disclosure, any limitations arising on account of the same are not intended.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, the order of processes described herein may be changed and are not limited to the manner described herein. Moreover, the actions of any flow diagram need not be implemented in the order shown; nor do all the acts need to be necessarily performed. Also, those acts that are not dependent on other acts may be performed in parallel with the other acts.

The scope of embodiments is by no means limited by these specific examples.

We claim:

1. A system for enabling medical consultation online comprising:
a processing subsystem hosted on a server and configured to execute on a network to control bidirectional communications among a plurality of modules comprising:
a registration module configured to register one or more health care professionals by creating one or more health care professional profiles in an integrated database upon receiving one or more health care professional details via one or more devices;
a specialist search module operatively coupled to the registration module, wherein the specialist search module is configured to conduct a search for a first health care professional in the one or more health care professional profiles created by the registration module upon receiving a search query raised by a second health care professional, wherein the search query comprises one or more parameters associated with the first healthcare professional;
an appointment management module operatively coupled to the specialist search module, wherein the appointment management module is configured to reserve an appointment of the first health care professional appeared in the search conducted by the specialist search module upon receiving a reservation request from the second health care professional;
a consultation module operatively coupled to the appointment management module 90, wherein the consultation module is configured to:
establish a two-way communication channel between the first health care professional and the second healthcare professional at a time specified during a reservation of the appointment by the appointment management module to avail a consultation of first health care professional by the second health care professional; and
provide one or more medical records of one or more patients relevant to the consultation from the integrated database to the first healthcare professional for consultation.
a diagnostic module operatively coupled to the integrated database, wherein the diagnostic module is configured to:
prepare a transcript of the consultation by analyzing one or more audio signals produced during the consultation of the first health care professional using at least one natural language processing technique upon identifying the one or more medical conditions;
analyze the one or more medical records of the one or more patients and the transcript using a plurality of image processing techniques to generate a first pattern;
compare the first pattern with a prestored data in the integrated database to identify one or more medical conditions of the one or more patients;
recommend one or more additional medical procedures to the one or more patients using a plurality of neural network techniques, wherein the one or more additional medical procedures comprises at least one of an acute disease screening, chronic diseases screening, immunization programs, cancer screening or a combination thereof.

2. The system as claimed in claim 1, wherein the one or more health care professional details comprises at least one of an enrollment ID, personal whereabouts, experience, specialization and available timings for consultation.

3. The system as claimed in claim 1, wherein the one or more parameters comprises at least one of a specialization, the available time for the consultation, an experience or a combination thereof.

4. The system as claimed in claim 1, wherein the processing subsystem comprises a payment management module configured to:
generate a payment link to the second health care professional to enable a payment of the predefined consultation fee;
enable the payment of the predefined consultation fee via the payment link through one or more payment gateways; and
generate a payment receipt upon successful payment of the predefined consultation fee and update the payment details corresponding to the one or more healthcare professional profiles.

5. The system as claimed in claim 1, wherein the processing subsystem comprises a reminder generation module operatively coupled to the integrated database, wherein the reminder generation module is configured to generate one or more alerts to remind the one or more health care professionals regarding the appointment based on the time specified during the reservation of the appointment by the appointment management module.

6. The system as claimed in claim 1, wherein the processing subsystem comprises a status update module operatively coupled to the integrated database, where in the status update module is configured to:
update one or more available timings for consultation by the one or more health care professionals in corresponding the one or more health care professional profiles; and
update a real time availability of the one or more healthcare professionals corresponding to the one or more health care professional profiles.

7. The system as claimed in claim 1, wherein the processing subsystem comprises an advertisement module operatively coupled to the integrated database, wherein the advertisement module is configured to display one or more advertisements on the one or more devices associated with one or more healthcare professionals upon receiving one or more targeting parameters from the integrated database and geo location data associated with the one or more healthcare professionals.

8. The system as claimed in claim 1, wherein the processing subsystem comprises a subscription management module operatively coupled to the integrated database, wherein the subscription management module is configured to provide controlled access for the one or more healthcare professionals to the plurality of the modules based on a subscription secured by the one or more healthcare professionals.

9. The method for enabling medical consultation online comprising:
registering, by a registration module, one or more health care professionals by creating one or more health care professional profiles in an integrated database upon receiving one or more health care professional details via one or more devices;
conducting, by a specialist search module, a search for a first health care professional in the one or more health care professional profiles created by the registration module upon receiving a search query raised by a second health care professional, wherein the search query comprises one or more parameters associated with the first healthcare professional;
reserving, by an appointment management module, an appointment of the first health care professional appeared in the search conducted by the specialist search module upon receiving a request of the second health care professional;
establishing, by a consultation module, a two-way communication channel between the first health care professional and the second healthcare professional at a time specified during a reservation of the appointment by the appointment management module to avail a consultation of first health care professional by the second health care professional;
providing, by the consultation module, one or more medical records of one or more patients relevant to the consultation from the integrated database to the first healthcare professional for consultation;
preparing, by a diagnostic module, a transcript of the consultation by analyzing one or more audio signals produced during the consultation by the first health care professional using at least one natural language processing technique upon identifying the one or more medical conditions;
analyzing, by the diagnostic module, the one or more medical records of the one or more patients and the transcript using a plurality of image processing techniques to generate a first pattern;
comparing, by the diagnostic module, the first pattern with a prestored data in the integrated database to identify one or more medical conditions of the one or more patients; and
recommending, by the diagnostic module, one or more additional medical procedures to the one or more patients using a plurality of neural network techniques, or prestored algorithms wherein the one or more additional medical procedures comprises at least one of an acute disease screening, chronic diseases screening, immunization programs, cancer screening or a combination thereof.
